(19) United States
(12) Patent Application Publication
(10) Pub. No.: US 2005/0282155 A1
(43) Pub. Date: Dec. 22, 2005

(54) Viral libraries from uncultivated viruses and polypeptides produced therefrom

(75) Inventors: Thomas William Schoenfeld, Madison, WI (US); David Alan Mead, Middleton, WI (US)

Correspondence Address: MICHAEL BEST & FRIEDRICH, LLP, ONE SOUTH PINCKNEY STREET, P O BOX 1806, MADISON, WI, 53701, US

(73) Assignee: Lucigen Corporation, Middleton, WI (US)

(21) Appl. No.: 11/152,883

(22) Filed: Jun. 15, 2005

Related U.S. Application Data
(60) Provisional application No. 60/580,515, filed on Jun. 17, 2004.

(51) Int. Cl. C12Q 1/70; C12Q 1/68

(52) U.S. Cl. 435/5; 435/6

(57) ABSTRACT

Provided is a method for producing viral genomic and complementary DNA expression libraries and novel viral polypeptides without requiring virus cultivation prior to library construction. The method includes direct isolation of viral particles from the environment by differential filtration and centrifugation. The viral nucleic acids are extracted and used to construct libraries that may be screened by one of several methods to identify useful coding sequences or may be used to produce novel polypeptides.

### CROSS REFERENCE TO RELATED APPLICATIONS

This application claims benefit of priority from U.S. Provisional Application Ser. No. 60/580,515, filed Jun. 17, 2004.

### STATEMENT REGARDING FEDERALLY SPONSORED RESEARCH

This invention was made with United States government support awarded by the National Science Foundation (Grant Nos. 0109756 and 0215988) and the National Institutes of Health (Grant No. 2R44 HG002714-02). The United States has certain rights in this invention.

### INTRODUCTION

Proteins derived from viruses are widely used in biotechnology, and many are economically valuable. Viral proteins, including viral enzymes, are valuable for use in research, diagnostics, and clinical applications, including DNA synthesis, nucleic acid sequencing, detecting mutations, synthesizing complementary DNA from RNA, and preparing DNA for molecular cloning, among others. Moreover, some viral proteins have potential applications as antimicrobial therapies.

Viruses are attractive sources of new enzyme activities, if a practical method of identifying and producing such enzymes were available. Viral abundance (the number of viruses on earth) and viral diversity (the number of different species) are believed to exceed those of all other life by at least ten-fold. Viral proteins of a given type are also very diverse at the amino acid level, suggesting that the proteins will have novel, potentially useful, activities or properties.

Despite the uses and advantages of viral enzymes, most viruses have not been screened as a source of new enzymes. Virtually all commercial enzymes are derived from a very small number of viruses that are relatively easily cultivated in the laboratory, notably bacteriophages T4, T7, T3, SP6, lambda, phi29, and eukaryotic viruses Avian Myeloblastosis Virus (AMV) and Moloney Murine Leukemia Virus (MMLV).

DNA polymerases are widely used as reagent enzymes. Enzymes of this class are used for detecting and isolating, with high sensitivity and selectivity, polynucleotide sequences among a mixture of other polynucleotides, determining the order in which individual nucleotides exist in specific polynucleotides, and modifying and manipulating polynucleotides for analysis and cloning. Among these enzymes, the most widely used DNA polymerases are those that are stable to greater than 90° C. and are active at temperatures of a least 70° C., conditions commonly used in DNA detection and analysis methods. However, no available viral DNA polymerases are stable and optimally active at greater than 70° C. In spite of years of effort by several independent groups to identify and express viral genes to produce thermostable DNA polymerases, only a few have been reported. The sole commercially available thermostable DNA polymerase is stable to only 55° C.

The traditional approach for identifying new viral enzyme coding sequences is inefficient because it requires culturing host cells, infecting the cells with virus, and identifying proteins expressed temporally after infection. This process typically requires extensive optimization of culture conditions for both the host and the virus. Testing for new enzyme activities typically takes months or years for each virus. Furthermore, this approach severely restricts the scope of viruses that can be screened to the very few that are amenable to culture by existing methods.

To circumvent similar difficulties with identifying enzymes derived from culture-resistant microbial cells, methods have been developed to identify coding sequences of uncultivated microorganisms. These methods involve isolating microorganisms from the environment, isolating the genetic material without the intermediate step of culturing the microbe, and using one of several methods to identify specific coding sequences, for example, specific types of enzymes. A common limitation of these methods is that they specifically prevent the isolation of viral genes. In fact, recent work to discover uncultured environmental microbial diversity has all but ignored phage and viral diversity in the environment.

It is appreciated that production of viral proteins directly from the original source organisms is problematic. Culturing of viruses remains technically difficult. Moreover, the expression of viral genes is often transient and dependent upon the selection of host and culture conditions. Typically, yields of viral proteins from infected cells or viral particles are much too low for commercial purposes. Like many cellular enzymes, viral enzymes and other gene products can be expressed economically in production hosts, but only after the coding sequence has been identified and cloned.

Identifying and cloning protein coding sequences from uncultivated viruses presents many challenges. Specifically, generating libraries that are useful sources of viral coding sequences from uncultured viral DNA is problematic, as viruses are present in the environment at very low concentrations compared to the concentration of cultured viruses typically available for library construction. For example, reported viral concentrations in aquatic environments range from 6,000 viruses per milliliter of water in the Sargasso Sea, a nutrient poor environment, to 300,000,000 viruses per milliliter in various estuaries and freshwater lakes. Measured viral concentrations in thermal environments range from 7,000 to 700,000 viruses per milliliter. The typical virus contains about 0.05 femtograms of DNA.

Using these numbers, the amount of DNA that theoretically can be isolated under perfect circumstances from aquatic environments ranges from 300 femtograms to 15 nanograms per milliliter, and the maximum amount of DNA that can be isolated from thermal environments ranges from 350 femtograms to 35 picograms per milliliter, although actual yields are generally much lower. Published library construction methods, however, require 10 micrograms of DNA. To obtain this amount, all the viruses must be isolated from between 0.67 and 33,000 liters of aquatic environment and between 290 to 29,000 liters of thermal environments. While published methods allow isolation of viruses from up to several liters, there are no published methods for constructing viral DNA libraries from thousands of liters.

Moreover, viral DNA is often modified with prosthetic chemical groups. These modifications can induce restriction systems inherent in common bacterial hosts. These restriction systems recognize modified DNA as foreign and prevent stable maintenance in the host cell by enzymatically degrading the foreign DNA.

### SUMMARY OF THE INVENTION

The approach described herein advantageously provides a method for producing viral genomic and expression libraries from diverse environments without the need for virus cultivation. The invention also provides a method of producing proteins encoded by viral inhabitants of extreme environments, including natural thermal environments such as hot springs. The presently described methods greatly increase the rate at which new viral enzyme coding sequences can be identified and greatly increase the breadth of virus-encoded proteins that can be produced. Implementation of these methods promises to allow introduction of new enzymes that are more suitable to various applications. For example, virally modified nucleotides are replaced with standard nucleotides in the libraries of the invention, permitting maintenance of viral DNA in host cells. Moreover, in accordance with the present invention, environmental phage and viruses discarded by currently practiced methods may be captured and concentrated. Accordingly, the invention provides access to a wider range of viral enzymes than previously practiced methods. In addition, the presently described methods are rapid and cost-effective.

In one aspect, the invention provides a method of constructing a viral expression library from uncultivated viruses. The method includes steps of substantially isolating viral particles from an environmental source, extracting viral polynucleotides from the viral particles, producing DNA fragments from the viral polynucleotides and constructing a viral expression library using the DNA fragments.

In another aspect, the invention provides a method of producing viral polypeptides from uncultivated viruses. The method includes constructing a viral expression library as above and inducing expression of viral polypeptides from the viral expression library.

In yet another aspect, the invention provides a method of constructing a viral genomic library from uncultivated viruses. The steps of the method include substantially isolating viral particles from an environmental source, extracting viral polynucleotides from the viral particles, producing DNA fragments from the viral polynucleotides, wherein at least about 90% of the DNA fragments are at least about 2 kb or greater, and constructing a viral genomic library using the DNA fragments.

In a further aspect, the invention provides a method of identifying viral polypeptides of interest. Steps in the method include substantially isolating viral particles from an environmental source, extracting viral polynucleotides from the viral particles, producing DNA fragments from the viral polynucleotides, constructing an expression library using the DNA fragments, inducing expression of viral polypeptides from the expression library and characterizing the viral polypeptides to identify viral peptides of interest.

Yet a further aspect of the invention provides a method of identifying polynucleotides encoding viral polypeptides of interest comprising substantially isolating viral particles from an environmental source, extracting viral polynucleotides from the viral particles, producing DNA fragments of at least about 2 kb from the viral polynucleotides, constructing a genomic library using the DNA fragments, screening the genomic library to identify polynucleotides encoding viral polypeptides of interest.

A further aspect of the invention provides kits for constructing a viral expression library from uncultivated viruses recovered from an environmental source. Such kits suitably include a filter capable of excluding particles sized from about 30 kD to about 300 kD, and a filter having a pore diameter of from about 0.10 micrometers to about 0.45 micrometers.

### DETAILED DESCRIPTION OF SEVERAL EMBODIMENTS

The invention provides methods of constructing both viral expression libraries and viral genomic libraries, from uncultivated viruses. An “uncultivated virus,” as the term is used herein, refers to a virus that is not maintained in a host cell under conditions suitable to the host cell. As used herein, an “expression library” refers to a vector-based collection of one or more polynucleotide molecules which encode one or more viral polypeptides, wherein the polynucleotide molecules are operably connected to a suitable active promoter from which the viral polypeptides may be expressed. A “genomic library,” as used herein and in the art, refers to a vector-based collection of one or more viral polynucleotide molecules derived directly or indirectly from virus particles.

Isolation of Viral Particles

As an initial step in several embodiments of the present invention, uncultivated viruses are substantially isolated from an environmental source. “Substantially isolating” refers to excluding non-viral matter from viruses using differential filtration and centrifugation techniques. As will be understood by the skilled artisan, the presence of trace amounts of contaminating microorganisms, cells, proteins, extracellular DNA and/or other debris in viral preparations isolated according to the invention does not remove such viral preparations from the scope of the appended claims.

In some embodiments, substantially isolating viral particles includes concentrating an environmental sample to enrich for viral particles using differential filtration techniques. “Differential filtration,” as used herein and in the art, refers to the practice of separating particles occupying different volumes using filters having different pore sizes. Other methods of “substantially isolating” viral particles (e.g., precipitation by polyethylene glycol) are known to those skilled in the art, and such methods are also contemplated herein.

The environmental source from which viruses are suitably isolated can be an aquatic source or a solid substrate. Suitable environments include, but are not limited to, aquatic environments (e.g., freshwater, marine, thermal springs, saline, acidic, or alkaline) or solid materials (e.g. soil, thermal mud, plant surfaces, lichens or wood). Viruses may be isolated from environments characterized by extremes of heat (e.g., thermal hot springs or thermal mud pots) or cold (e.g., a cold seep or Antarctic ice or ice melt). Using the presently described methods, viruses may also be isolated from environments characterized by other extreme conditions, such as extremes of pH or salinity. Alternatively, viruses may be isolated from animal sources, including human sources, such as samples from the digestive tract, skin or other tissues, or body fluid such as blood or urine.

Viruses collected from environmental sources are suitably isolated and concentrated by a series of filtrations and centrifugations. In one embodiment, the viral component of aquatic environments may be suitably concentrated by ultrafiltration using a suitable molecular weight cut-off tangential flow filter. One such suitable filter is A/G Technology model UFP-100-C9A, having a 100 kD molecular weight cut-off (Amersham Bioscience). One skilled in the art will recognize that similar filtration systems having molecular weight cut-off pore sizes of about 30 kD to about 300 kD would also be suitable for use in the present invention. Viral particles can be concentrated to a final volume of 2 liters or less from an initial sample of up to 1500 liters by this method. Collection of the filtrate from a second filtration step using a filter having a pore size of about 0.45 μm results in a viral concentrate substantially free of cells and other debris. One skilled in the art will recognize that filters having pore sizes of about 0.05 μm to about 0.85 μm would also be suitable. More suitably, the filters may have pores sized from about 0.1 μm to about 0.45 μm or to about 0.65 μm. The concentrate will contain viral particles, largely free of extracellular DNA, environmental cells and debris.

In a further embodiment of the invention, the viral component of a solid environmental sample is isolated and concentrated. In one suitable method, suspended viruses are separated from the soil particles by centrifugation at, e.g., 12,000×g for 30 minutes, although centrifugation at higher or lower speeds (e.g. 4,000 to 60,000×g) or for shorter or longer times (e.g., 5 min. or longer) would also be expected to be effective. The supernatant, which contains viral particles, is recovered. Undesired larger particles such as soil particles are suitably removed by filtration through a nylon mesh of 100 μm, although meshes ranging from 1 μm to 1 mm may be used. The resulting extract is suitably filtered by a 0.2 μm filter and concentrated to, e.g., 100 ml using a suitable filter as described above. Filters in a range of 0.05 μm to 0.65 μm are expected to be useful as well.

Additional rounds of filtration and centrifugation may be used subsequently to further isolate viruses and/or concentrate the virus preparation and thereby improve the yield of transformants and reduce the background of nonviral sequences. Suitable filters have pore sizes of about 0.1 μm to about 0.45 μm. Use of these filters provide filtrates that are highly enriched for viral particles and extracellular DNA and can be further concentrated to a volume of 100 ml, although volumes from 1 ml to several liters would also be effective, using, e.g., a 100 kD molecular weight cut-off filter. Viral particles are suitably enumerated and the presence or absence of cells is detected by use of standard epifluorescence microscopy methods. A small, but detectable, number of cells may contaminate the filtrates. Such cellular contamination, if present, may be reduced by still additional rounds of filtration and centrifugation as described above. Preferably, resulting viral preparations are substantially free of microbial and other cellular contaminants.

Other methods of viral isolation and concentration known in the art are also potentially suitably used in the present invention. For example, viral particles can be pelleted by centrifugation at 100,000×G and resuspended in an appropriate buffer. Alternatively, viral particles may be concentrated to a volume of about 200 μl and buffer exchanged by use of a 100 kD molecular weight cut-off spin filter or similar filters. One skilled in the art will also appreciate that methods commonly used to purify cultured phage will also be effective. Such methods typically include precipitation with polyethylene glycol, or step-gradient or isopycnic equilibrium gradient ultracentrifugation in the presence of an appropriate solute, generally cesium chloride or sucrose. Viruses are also suitably purified by ion exchange chromatography, methods for which are described in the art.

The free DNA that contaminates viral particles is suitably removed by incubation with an appropriate nuclease or combination of nucleases. As will be understood, encapsidated viral DNA is protected from nuclease treatment. After incubation with nucleases, the concentrated viral particles are substantially free of contaminating DNA of any kind. The nuclease is suitably inactivated by addition of EDTA prior to extraction of viral polynucleotides.

Extraction of Viral Polynucleotides

In a further step of some embodiments of the present methods, viral polynucleotides are extracted from the isolated viral preparation. The isolated viral preparation can be expected to include populations of both DNA and RNA viruses. Accordingly, “viral polynucleotides,” include both DNA and RNA molecules and also include both single-stranded (including plus-sense and minus-sense) and double-stranded molecules. Reference to particular viral polynucleotides is intended to encompass both DNA sequences as well as RNA corresponding to the DNA sequences, and also includes complementary sequences.

The viral polynucleotides are suitably extracted according to conventional methods. For example, viral DNA can be extracted by incubation with 0.5% SDS detergent and 0.5 mg/ml proteinase K at 56° C. for 1 hour. The viral DNA is recovered by standard methods, for example by using cetyltrimethylammonium bromide (CTAB)/chloroform extraction, followed by a standard phenol:chloroform extraction and alcohol precipitation. As will be appreciated by the skilled artisan, viral RNA can be extracted using a similar process. In one standard method, after proteinase K digestion, a solution of guanidinium thiocyanate, sodium citrate, sodium lauryl sarcosinate and mercaptoethanol is added to the viral preparation and the suspension is mixed thoroughly. Sodium acetate is then added to the solution. Viral RNA can be extracted with phenol:chloroform and precipitated with isopropanol.

As will be understood, other methods of extracting viral polynucleotides from whole virus preparations are also commonly practiced in the art and are suitable for use in the present invention.

Production of DNA Fragments from Extracted Viral Polynucleotides

In a subsequent step, DNA fragments are suitably produced from the viral polynucleotides. The term “DNA fragments,” as used herein and in the art, refers to two or more DNA molecules produced from one or more larger DNA molecules. DNA fragments may be genomic DNA or complementary DNA generated from genomic RNA molecules. If the extracted polynucleotides are RNA molecules, complementary DNA (cDNA) is suitably synthesized according to standard methods for use in library construction. For example, first strand cDNA can be synthesized from genomic RNA by MMLV reverse transcriptase using random hexamer primers. The second strand can be suitably synthesized using E. coli DNA Pol I and T4 DNA Pol priming at nicks generated by RNase H. The cDNA product can be isolated, e.g., by phenol/chloroform isolation followed by alcohol precipitation.

In some embodiments of methods of constructing viral genomic or expression libraries, purified viral DNA or cDNA produced from viral RNA is sheared to generate DNA fragments. “Shearing,” as used herein and in the art, refers to mechanical, enzymatic or chemical disruption of a polynucleotide to form smaller polynucleotide fragments. Various methods of shearing are known in the art, including using a shearing instrument such as a HYDROSHEAR® device (Gene Machines), partial digestion by restriction endonuclease or other nuclease, sonication or nebulization. It will be appreciated that shearing or other methods of producing DNA fragments generate a population of fragments of varying lengths. Suitably, at least about 90% of the fragments generated are at least about 2 kb or greater. More suitably, at least about 90% of the fragments generated are from about 2 kb to about 10 kb in length. Moreover, fragments as large as entire viral genomes may function in constructing libraries as well.

Amplification of DNA Fragments

If at least about one μg of viral DNA or cDNA is generated from viral RNA, libraries may be constructed directly, although successful library construction generally requires about 10 μg DNA or cDNA. Accordingly, the sheared viral DNA optionally may be amplified prior to construction of the expression library. “Amplifying,” as used herein and in the art, refers to the molecular generation of polynucleotide molecules identical to a template polynucleotide molecule. Amplification is suitably carried out using molecular cloning techniques commonly used in the art, including PCR.

Construction of Viral DNA Libraries

Expression and/or genomic libraries are suitably constructed from the amplified or unamplified DNA fragments. As used herein, an “expression library” refers to a vector-based collection of one or more polynucleotide molecules which encode one or more viral polypeptides, wherein the polynucleotide molecules are operably connected to a suitable active promoter from which the viral polypeptides may be expressed. A polynucleotide is “operably linked” or “operably connected” when it is placed into a functional relationship with another polynucleotide element. For instance, a promoter or enhancer is operably linked to a coding sequence if it affects the transcription of the coding sequence. Operably linked sequences are typically contiguous and, where necessary to join two protein coding regions, contiguous and in reading frame. An “active promoter,” is a nucleotide sequence to which an RNA polymerase binds to initiate transcription. A promoter is active if it is compatible with host cell RNA polymerases, allowing for synthesis of RNA. Typical promoters used in eukaryotic expression systems include, but are not limited to, CMV immediate early, HSV thymidine kinase, early and late SV40, LTRs from retroviruses and mouse metallothionein 1. Suitable bacterial promoters include lacI, lacZ, T3, T7, gpt, lambda PR, PL and trp. Selection of an appropriate promoter is well within the level of ordinary skill in the art. Moreover, as will be appreciated, homolgous viral sequences isolated in accordance with the invention may function as “active promoters” within the viral libraries, and are therefore are included in the scope of the definition.

A “genomic library,” as used herein and in the art, refers to a vector-based collection of one or more viral polynucleotide molecules derived directly or indirectly from virus particles, and which may or may not include promoter sequences.

Suitable means of constructing viral genomic or expression libraries from a population of DNA fragments are known. Suitably, viral DNA or cDNA fragments generated as described above are enzymatically treated to generate blunt, phosphorylated termini. Double-stranded oligonucleotides (linkers) are ligated to the ends of the sheared DNA. An example of suitable asymmetric linker sequences of 28 and 30 nucleotides are shown below, although it will be appreciated that numerous additional sequences would also function as well.

| | |
|---|---|
| 5′-GGAGCAGTATCAGATACAAGCGGCCGCATC-3′ | (SEQ ID NO:1) |
| 3′-TCGTCATAGTCTATGTTCGCCGGCGTAG-5′ | (SEQ ID NO:2) |

The ligation products are then suitably size-fractionated and excess linkers and smaller fragments that may result from the shearing procedure are removed according to standard methods, e.g., by separation techniques such as agarose gel electrophoresis. The linker-ligated fragments are recovered and the DNA fragments between the linkers are suitably amplified by a suitable DNA polymerases, e.g., a thermostable DNA polymerase, and oligonucleotide primers that hybridize to the linkers.

Linker-ligated, amplified or non-amplified DNA sequences are then suitably ligated into cloning vectors by methods commonly practiced by those skilled in the art to provide a recombinant DNA fragment. As used herein, a “recombinant DNA fragment” is a DNA sequence formed by combining two non-homologous DNA molecules, e.g., viral DNA and a cloning vector. Suitably, the cloning vectors are plasmid vectors. Plasmid vectors suitable for use in the invention can be of two types: 1) those with active promoters (expression vectors) that allow clones to be directly screened based on expression of the inserted DNA, by genetic homology, or by complementation, or 2) those lacking active promoters (non-expression vectors), in which clones can be screened based on polynucleotide sequence homology. Alternatively, phage-derived vectors or other suitable cloning vehicles may also be used.

In some embodiments, suitable hosts are transformed with the recombinant DNA fragments to propagate genomic or expression libraries for further analysis or for expression of viral polypeptides. Suitable hosts include prokaryotic and eukaryotic cells. One suitable prokaryotic cell is E. coli, however, any bacterial cell may be selected for use in the methods of the invention by the ordinarily skilled artisan. Suitable eukaryotic cells which may be used include mammalian cells and yeast cells.

Production of Viral Polypeptides

In some cases, it may be desired to produce viral polypeptides from uncultivated viruses. In these instances, expression of viral polypeptides is induced from viral expression libraries constructed as described above. “Inducing expression of viral polypeptides,” as used herein and in the art, refers to initiating gene expression to produce protein. Inducing may involve the activation of inducible promoters (e.g., by contacting host cells with a chemical inducer such as Isopropyl-beta-D-thiogalactopyranoside (IPTG) or manipulation of the incubation temperature), or the passive induction of constitutive promoters. Expressing viral polypeptides from host cells is well within the capabilities of the skilled artisan.

As will be appreciated, “viral polypeptides,” as used herein and in the art, include both full-length proteins and peptides encoded by expression libraries according to the invention, as well as fragments thereof. Proper folding or subunit aggregation or association may or may not occur in expressed proteins.

Identification of Viral Polypeptides of Interest and/or Viral Polynucleotides Encoding Polypeptides of Interest

The viral libraries produced according to the invention can be screened by one of several methods to detect the rare occurrence of coding sequences for a novel member of a group of valuable proteins, such as a specific class of enzymes.

One suitable screening method is homology screening, in which the nucleotide sequence is determined and conceptually translated to an amino acid sequence using commercially or publicly available software, such as BLASTx (NCBI). The nucleotide sequence or the inferred amino acid sequence are compared to nucleotide sequences or amino acid sequences in publicly available databases.

Another common method of detecting similarity of an unknown sequence to a sequence of known function is hybridization of the unknown sequence to a polynucleotide probe based on the known sequence. Similarity of the viral DNA or protein sequence to sequences of known function is generally regarded as evidence of shared function.

Expression of viral polypeptides from, e.g., expression libraries permits characterization of the viral polypeptides to identify viral polypeptides of interest. As used herein, “characterizing viral polypeptides” refers to the use of any assay to identify viral polypeptides, and includes quantitative, qualitative and functional assays.

In expression screening, the presence of coding sequences is inferred by the detection of the expression of polypeptides in clones harboring the coding sequences. Expression can be detected by various methods. Expression of polypeptides with enzymatic activity can be detected in libraries of the clones by assays for enzyme activity.

Alternatively, expression of proteins can be detected by immunoscreening, in which the expressed proteins are tested for cross reactivity to antibodies raised against proteins of interest. Evidence of cross reactivity is generally regarded as implying shared three dimensional structure and, therefore, shared function.

Yet another suitable screening method is known as complementation screening. In this method, the libraries can be used to transform a host that is mutant in a given gene of interest. The reversion or repression of the mutant phenotype to a phenotype more like that of wild type due to foreign DNA is generally regarded as implying that the cloned DNA at least partially shares a function with the mutant gene.

In a further suitable screening method, polynucleotides encoding polypeptides of interest can be identified by genetic selection in which culture conditions are designed so that the chances of survival of a host cell or the maintenance of a recombinant vector:insert complex are enhanced by the polypeptide expressed.

In particularly suitable embodiments, viral DNA polymerases are identified and characterized. A “DNA polymerase”, as used herein and in the art, refers to enzymes capable, in the presence of a primed deoxyribonucleic acid template, of incorporating individual deoxyribonucleotides into polynucleotides in a specific order dictated by the template. DNA polymerases are suitably identified by demonstration of DNA polymerase activity. “DNA polymerase activity,” “synthetic activity” and “polymerase activity” are used interchangeably herein and in the art to refer to the ability of an enzyme to synthesize new DNA strands by the incorporation of deoxynucleoside triphosphates. A protein that can catalyze the synthesis of new DNA strands by the incorporation of deoxynucleoside triphosphates in a template-dependent manner is said to be “capable of DNA synthetic activity.”

Kits

In some embodiments, the invention provides kits that may be used to construct viral libraries in accordance with the invention. Such kits may contain any combination of suitable filters, including, e.g., a filter capable of excluding particles sized from about 30 kD to about 300 kD and a filter having a pore diameter of from about 0.10 μm to about 0.45 μm. A useful filter for inclusion in kits has a pore diameter of about 0.20 μm. Suitably, the filters are packaged together and the kit further comprises instructions for using the filters to isolate viral particles from an environmental source.

Optional further components of kits of the invention include one or more prokaryotic or eukaryotic host cells, preferably a culture of host cells such as E. coli cells, and a cloning vector having a promoter active in the host cells.

The present invention may be better understood with reference to the following examples. These examples are intended to be representative of specific embodiments of the invention, and are not intended to limit the reasonable scope thereof.

### EXAMPLES

### Example 1

### Construction of a Sequencing Library from Viral DNA

Isolation of Uncultured Viral Particles from a Thermal Spring

Viral particles were isolated from a thermal spring in the River Group of the Lower Geyser Basin of Yellowstone National Park (N 44.5610, W 110.8337, temp. 82° C., pH 7). This thermal spring is commonly known as Azure Hot Spring. Thermal water was filtered using a 100 kiloDalton mwco tangential flow filter (A/G Technology, Amersham Biosciences) at the rate of 5 liters per minute over 5 hours (1500 liters overall), and viruses and microbes were concentrated to 2 liters. The resulting concentrate was filtered through a 0.2 μm tangential flow filter to remove microbial cells. The viral fraction was further concentrated to 100 ml using a 100 kD mwco tangential flow filter. Of the 100 ml viral concentrate, 40 ml was processed further. Viruses were further concentrated to 400 μl by filtration in a 30 kD mwco spin filter (Centricon, Millipore).

Isolation of Viral DNA

The viruses were transferred to SM buffer (0.1 M NaCl, 8 mM MgSO₄, 50 mM Tris HCl 7.5) and concentrated to 400 μl in a 30 kD mwco spin filter (Centricon, Millipore). Endonuclease (Sigma, 10 U) was added to remove non-encapsidated (non-viral) DNA. The reaction was incubated for 30 min. at 23° C. Subsequently, EDTA (20 mM) and sodium dodecyl sulfate (SDS) (0.5%) was added. To isolate viral DNA, Proteinase K (100 U) was added and the reaction was incubated for 3 hours at 56° C. Sodium chloride (0.7M) and cetyltrimethylammonium bromide (CTAB) (1%) were added. The DNA was extracted once with chloroform, once with phenol, once with a phenol:chloroform (1:1) mixture and again with chloroform. The DNA was precipitated with 1 ml of ethanol and washed with 70% ethanol. The yield of DNA was 300 ng.

Construction of a Viral DNA Library

Viral DNA (10 ng) from above was physically sheared to between 2 and 4 kilobases (kb) using a HYDROSHEAR® Device (Gene Machines). These fragments were ligated using standard methods to double-stranded linkers of the following sequences:

| | |
|---|---|
| 5′-GGAGCAGTATCAGATACAAGCGGCCGCATC-3′ | (SEQ ID NO:1) |
| 3′-TCGTCATAGTCTATGTTCGCCGGCGTAG-5′ | (SEQ ID NO:2) |

The ligation mix was separated by agarose gel electrophoresis and fragments in the size range of 2-4 kb were isolated. These fragments were amplified by standard PCR methods.

The amplification products were inserted into the cloning site of pSMART vector (Lucigen, Madison, Wis.) and used to transform E. cloni 10 G cells (Lucigen, Madison, Wis.).

Screening by Homology

Clones were isolated and the sequences of their inserts were determined by standard methods. The sequences were conceptually translated and compared to the database of non-redundant protein sequences in GenBank (NCBI) using the BLASTx program (NCBI). One of these sequences had significant similarity to several dozen sequences of bacterial DNA polymerase III alpha subunit. The E (expect) values ranged down to 3⁻¹⁰, indicating a very high probability that the sequence is that of an authentic DNA polymerase gene.

### Example 2

### Construction of an Expression Library from Viral DNA

Isolation of Uncultured Viral Particles from a Thermal Spring

Viral particles were isolated from a thermal spring in the White Creek Group of the Lower Geyser Basin of YNP (N 44.53416, W 110.79812 temp. 80° C., pH 8), commonly known as Octopus Spring. Thermal water was filtered as described at the rate of 7 liters per minute over 90 min. (630 liters overall), and viruses and microbes were concentrated to 2 liters. The resulting concentrate was filtered to remove microbes and further concentrated to 400 μl as described.

Isolation of Viral DNA

The viral DNA was isolated as described in Example 1. The yield of DNA was 20 ng.

Construction of a Viral DNA Library

Viral DNA (20 ng) from above was physically sheared. These fragments were ligated to linkers as described above. The ligation mix was separated by agarose gel electrophoresis, and fragments in the size range of 2-4 kb were isolated and amplified and gel fractionated again. These fragments were inserted into the cloning site of pCR-SMART vector (Lucigen, Madison, Wis.) and used to transform BL21/DE3 cells (Novagen). The pCR-SMART vector contains a T7 promoter upstream from the insert to allow inducible expression.

Expression of Viral Genes

The cell clones were isolated and cultured overnight. The expression of cloned genes was induced by addition of IPTG. Protein expression was detected by SDS PAGE.

### Example 3

### Isolation of Viral DNA from Soil

Soil from two thermal mudpots, 760 and 80° C., collected at Hot Creek Gorge Thermal Area (N 37.66151, W 118.83015) was used to isolate viruses. Forty grams of soil suspension was centrifuged at 12,000×G for 30 min. to pellet contaminating cells and solid material. The filtration of the suspension through a 0.2 μm filter resulted in a viral concentrate substantially free of microbial contamination. The viral particles were exchanged to SM buffer and concentrated to 200 μl with a Centricon column. The viral suspension was digested with 10 U endonuclease (Sigma) for 1 hour at 23° C. EDTA (20 mM), NaCl (0.7 M), CTAB (1%), and Proteinase K (100 μg) were added. The reaction was incubated for 1 hour at 56° C. The viral DNA was isolated by choloroform extraction, followed by standard phenol/chloroform extraction and ethanol precipitation. The DNA was further purified using a DNA CLEAN AND CONCENTRATOR™ Kit (ZymoResearch). The yield of DNA was 170 and 185 ng for the two mudpot samples, respectively.

### Example 4

### Isolation of Viral DNA by Centrifugation

Viral particles were isolated from a thermal spring in the Lower Geyser Basin of YNP (N 44.33639, W 110.50045, 76° C.), commonly referred to as Cavern Spring, and processed to remove the bulk of microbial cells as described above. Thirty two ml of viral concentrates was centrifuged at 10,000×G for 30 min. to remove microbial cells. The viral concentrate was further purified using a 0.2 μm syringe filter. The viral particles were pelleted by ultracentrifugation at 100,000 g for 4 hours. The pellets were resuspended in 500 μl of TM Buffer (50 mM Tris HCl, pH 7.5, 8 mM MgSO₄). Endonuclease (5 U) was added, and the reaction was incubated for 1 hour at 23° C. To stop the reaction, EDTA (18 mM), SDS (0.5%) and Proteinase K (0.1 mg) were added. The reaction was incubated for 1 hour at 56° C. NaCl was added to 0.2M and the viral DNA was isolated by phenol/chloroform extraction and isopropanol precipitation. The DNA was suspended in 20 μl of water. The yield of viral DNA was approximately 10 ng.

### Example 5

### Identification, Expression and Production of a Functional Enzyme from a Viral Library

A library was constructed by methods described above from a fourth thermal spring in Yellowstone National Park (N 44.55617, W 110.83481, 75° C.). To our knowledge, this spring has no common name. Sequence analysis of this library identified a clone with strong sequence similarity to several known DNA polymerase-encoding genes, particularly Aquifex aeolicus polA (E value of 10-54 for the entire apparent coding sequence). This clone was cultured, the cells were lysed and proteins were extracted by standard methods. The extracted protein was incubated at 70° C. for 10 minutes to inactivate host enzymes. The remaining soluble protein was tested for DNA polymerase activity at 70° C. in a standard assay buffer. The assay used extension of fluorescently-labeled primer on a synthetic deoxyribonucleic acid template so that extension of the primer could be detected as a mobility shift on the laser-based capillary of an Applied Biosystems 310 Genetic Analyzer. The mobility shift corresponding to the predicted extension of the primer from 37 to 41 nucleotides indicated the presence of thermostable DNA polymerase activity.

As used in this specification and the appended claims, the singular forms “a,” “an,” and “the” include plural referents unless the content clearly dictates otherwise. Thus, for example, reference to a composition containing “a polynucleotide” includes a mixture of two or more polynucleotides. It should also be noted that the term “or” is generally employed in its sense including “and/or” unless the content clearly dictates otherwise. All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

The invention has been described with reference to various specific embodiments and techniques. However, it should be understood that many variations and modifications may be made while remaining within the spirit and scope of the invention.

1) A method of constructing a viral expression library from uncultivated viruses comprising:
a) substantially isolating viral particles from an environmental source;
b) extracting viral polynucleotides from the viral particles;
c) producing DNA fragments from the viral polynucleotides; and
d) constructing a viral expression library using the DNA fragments.

2) The method of claim 1, wherein substantially isolating viral particles further comprises concentrating the viral particles from a sample of the environmental source.

3) The method of claim 1, wherein the environmental source comprises extreme conditions.

4) The method of claim 1, wherein the environmental source is a thermal spring.

5) The method of claim 1, wherein the environmental source is a thermal mudpot.

6) The method of claim 1, wherein isolating the viral particles comprises use of a filter capable of excluding particles sized from about 30 kD to about 300 kD.

7) The method of claim 2, wherein concentrating the viral particles comprises use of a filter having a pore diameter of about 0.1 micrometers to about 0.45 micrometers.

8) The method of claim 1, wherein substantially isolating viral particles further comprises centrifugation of a sample from the environmental source.

9) The method of claim 1, wherein the viral polynucleotides comprise DNA.

10) The method of claim 1, wherein the viral polynucleotides comprise RNA.

11) The method of claim 10, wherein producing DNA fragments from the viral polynucleotides comprises reverse transcription of viral RNA polynucleotides.

12) The method of claim 1, wherein producing DNA fragments from the viral polynucleotides comprises shearing the viral polynucleotides.

13) The method of claim 1, further comprising amplifying the DNA fragments produced in step c).

14) The method of claim 1, wherein at least about 90% of the DNA fragments are at least about 2 kb or greater.

15) The method of claim 1, wherein constructing a viral expression library comprises operably connecting a DNA fragment to a promoter in a cloning vector to provide a recombinant DNA fragment.

16) The method of claim 15, further comprising transforming a host cell with the recombinant DNA fragment.

17) A method of producing viral polypeptides from uncultivated viruses from an environmental source comprising:
constructing a viral expression library according to the method of claim 1; and
inducing expression of viral polypeptides from the viral expression library to produce viral polypeptides.

18) A method of constructing a viral genomic library from uncultivated viruses comprising:
a) substantially isolating viral particles from an environmental source;
b) extracting viral polynucleotides from the viral particles;
c) producing DNA fragments from the viral polynucleotides, wherein at least about 90% of the DNA fragments are at least about 2 kb or greater; and
d) constructing a viral genomic library using the DNA fragments.

19) The method of claim 18, wherein substantially isolating viral particles comprises concentrating the viral particles from a sample of the environmental source.

20) The method of claim 18, wherein substantially isolating viral particles further comprises centrifugation of a sample from the environmental source.

21) A method of identifying viral polypeptides of interest comprising:
a) substantially isolating viral particles from an environmental source;
b) extracting viral polynucleotides from the viral particles;
c) producing DNA fragments from the viral polynucleotides;
d) constructing an expression library using the DNA fragments;
e) inducing expression of viral polypeptides from the expression library; and
f) characterizing the viral polypeptides to identify viral peptides of interest.

22) The method of claim 21, wherein characterizing the viral polypeptides comprises screening the viral polypeptides for enzyme activity.

23) The method of claim 21, wherein screening the viral polypeptides comprises expression screening, immunoscreening, complementation screening or genetic selection.

24) A method of identifying polynucleotides encoding viral polypeptides of interest comprising:
a) substantially isolating viral particles from an environmental source;
b) extracting viral polynucleotides from the viral particles;
c) producing DNA fragments of at least about 2 kb from the viral polynucleotides;
d) constructing a genomic library using the DNA fragments;
e) screening the genomic library to identify polynucleotides encoding viral polypeptides of interest.

25) The method of claim 24 wherein screening the genomic library comprises homology screening or hybridization screening.

26) A kit for constructing a viral library from uncultivated viruses recovered from an environmental source comprising:
a) a filter capable of excluding particles sized from about 30 kD to about 300 kD; and
b) a filter having a pore diameter of from about 0.10 micrometers to about 0.45 micrometers.

27) The kit of claim 26, further comprising:
c) one or more host cells; and
d) a cloning vector comprising a promoter active in the host cells.

28) The kit of claim 27, wherein the host cells are prokaryotic cells.

29) The kit of claim 27, wherein the host cells are eukaryotic cells.


SEQUENCE LISTING (2 sequences)

SEQ ID NO: 1 - DNA, 30 bp, Artificial sequence
  linker sequence
ggagcagtatcagatacaagcggccgcatc

SEQ ID NO: 2 - DNA, 28 bp, Artificial sequence
  linker sequence
tcgtcatagtctatgttcgccggcgtag